(19) United States
(12) Patent Application Publication
(10) Pub. No.: US 2003/0125680 A1
(43) Pub. Date: Jul. 3, 2003

(54) Hydrocolloid bandage

(76) Inventor: Jian Ling Ding, Glastonbury, CT (US)

Correspondence Address: GREENBERG TRAURIG LLP, SUITE 400E, 2450 COLORADO AVENUE, SANTA MONICA, CA, 90404, US

(21) Appl. No.: 10/335,047

(22) Filed: Dec. 31, 2002

Related U.S. Application Data
(60) Provisional application No. 60/345,796, filed on Jan. 2, 2002.

(51) Int. Cl. A61F 13/00

(52) U.S. Cl. 604/304; 602/54

(57) ABSTRACT

A hydrocolloid bandage for the wound care designed for improved pliability and decreased peripheral edge lift. In particular, the invention is concerned with a hydrocolloid bandage which is absorbent, non-damaging to the skin and comfortable to the user preferably having at least a hydrocolloid region covered by film and adhesive layers which contact the user's skin at the periphery of the bandage.

### RELATED APPLICATIONS

This application claims priority to U.S. Provisional Application Serial No. 60/345,796, filed on Jan. 2, 2002, herein incorporated by reference in its entirety.

### BACKGROUND OF THE INVENTION

This invention relates to a hydrocolloid bandage for use in wound care. In particular, the invention is concerned with a hydrocolloid bandage which is absorbent, non-damaging to the skin and comfortable to the user. Further, the invention is concerned with a bandage that is pliable and able to contour to the skin of the user and offers good adhesion to skin so that the hydrocolloid bandage does not lift from the skin during use.

Wound care is desirable to improve the health and appearance of underlying dermal tissues. Wounds, either injury induced, such as cuts, abrasions or blisters, or surgically induced, such as surgical incisions or ostomies, for example, require localized treatment to remedy the affected area and to prevent further dermal damage. If wounds are not properly treated, further dermal irritation can occur resulting in secondary infections and further discomfort to the patient.

Recently, the use of hydrocolloid bandages has spread beyond just the hospital setting and are now commonly found at the retail level for general consumer use. Although hydrocolloid bandages are more expensive than traditional bandages, their expense is justified by their improved utility for wound care. For example, hydrocolloid bandages 1) absorb moisture and wound extrudate, maintaining a moist environment and promoting wound healing; 2) remove excess liquid and perspiration from the skin surface to prevent maceration that can compromise the integrity of skin; 3) provide mild bonds to skin, which avoids physical irritation to skin upon dressing removal; 4) offers a long term wearing such as days, even a week.

However, there are limitations of the current hydrocolloid bandages when they are worn for a long period of time. A thick bandage profile makes it less conformable, and body movement can cause debonding and channeling of the bandage. The stress applied to the bandage dressing edge during a long term wearing can also cause edge lifting and/or channeling type of debonding from skin, which lead to adhesion failure. Also, the rubber PSA ingredients in the hydrocolloid matrix is prone to cold flow (flow at room temperature). The sticky adhesive tends to flow out of the bandage periphery, and cause sticky edges. Such sticky edges then catch clothes, bed sheets, etc. and cause edge lifting of the bandage.

Therefore, there is a need for a hydrocolloid dressing which provides good absorbency and/or good structural integrity, enhanced patient comfort and better adhesion to skin. Further, there needs to be a more conformable bandage with improved peripheral adhesion to skin, and decreased tendency to cold flow.

### SUMMARY OF THE INVENTION

The invention provides a hydrocolloid bandage which has improved advantages over known hydrocolloid dressings. The hydrocolloid bandages provide enhanced absorbency, enhanced structural integrity and enhanced adhesion to the wound site and patient comfort.

According to the invention, there is provided a hydrocolloid bandage (bandage) for wound care having an upper surface area and a lower surface area for adhering to the epidermis, dermis or wound area (skin) of the user.

The bandage preferably comprises a hydrocolloid region covered by a flexible film layer (film). Further, the bandage preferably includes a continuous or discontinuous adhesive layer. Optionally, a release liner can be applied to the bandage lower surface area to facilitate protection of the adhesive and/or hydrocolloid before application of the bandage to the user, for example.

In some embodiments where the film extends beyond the hydrocolloid region, skin adhesion is enhanced over currently available bandages. In other embodiments where an adhesive layer is attached to the film lower surface area, skin adhesion is also enhanced over currently available bandages. The film and adhesive extension is advantageous at least in eliminating the sticky hydrocolloid edge being outside of the bandage end due to the cold flow of hydrocolloid material. Further, the pressure sensitive adhesive under the film lower surface is advantageous in improving bandage bond to the skin as compared to hydrocolloid material alone. The adhesive secures the bandage end to skin better than a hydrocolloid dressing without such an adhesive coated extension.

The bandage can be made in a variety of shapes. However, the bandage preferably dimensioned in height, width and depth so as to provide enhanced absorbency, enhanced structural integrity and enhanced adhesion and patient comfort.

The foregoing and other objects, features, and advantages of the present invention will be apparent from the following detailed description of the preferred embodiments which makes reference to several drawing figures.

### BRIEF DESCRIPTION OF THE DRAWINGS

FIG. 1 is a perspective view of the components of one embodiment of the bandage being applied to the skin.

FIG. 2 is a perspective view of an alternate embodiment of the bandage.

FIG. 3 is a perspective view of another alternate embodiment of the bandage.

FIG. 4 is a perspective view of another alternate embodiment of the bandage.

FIG. 5 is a perspective view of the components of one embodiment of the bandage being applied to the skin.

FIG. 6 is a perspective view of an alternate embodiment of the bandage.

FIG. 7 is a perspective view of another alternate embodiment of the bandage.

FIG. 8 is a perspective view of another alternate embodiment of the bandage.

FIG. 9 is a view of the bandage upper surface area and method of applying the bandage.

### DETAILED DESCRIPTION OF THE PREFERRED EMBODIMENTS

In the following description of the preferred embodiments reference is made to the accompanying drawings which form the part thereof, and in which are shown by way of illustration of specific embodiments in which the invention can be practiced. It is to be understood that other embodiments can be utilized and structural and functional changes can be made without departing from the scope of the present invention.

Hydrocolloid Bandage

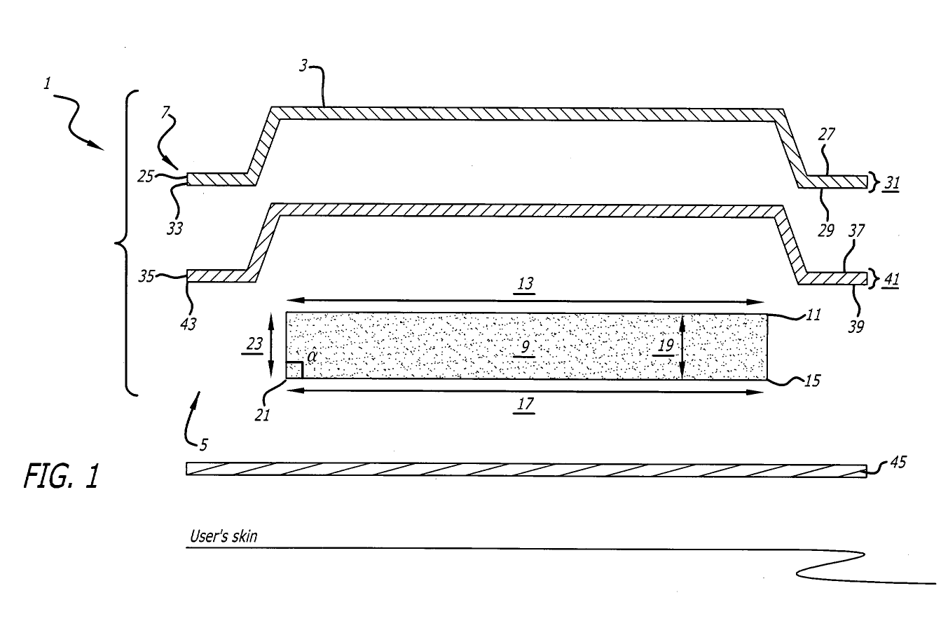

The hydrocolloid bandage (bandage 1) is for the treatment and/or protection of a wound when applied to the skin of the user (FIG. 1). As illustrated in FIG. 1, when applied to the user the bandage 1 has a bandage upper surface area 3 facing away from the user's skin and a bandage lower surface area 5 facing toward the user's skin. The bandage 1 is further defined in having a bandage end 7.

The bandage 1 generally is comprised of a hydrocolloid region 9 having a hydrocolloid upper surface area 11 having a length 13, hydrocolloid lower surface area 15 having a length 17, and a hydrocolloid thickness 19. The hydrocolloid thickness may range from about 3-100 mils, and most preferably about 20-65 mils. The hydrocolloid 9 is further defined in having a hydrocolloid end 21 having a length 23, extending at an angle α between the hydrocolloid lower surface area to the hydrocolloid upper surface area.

The hydrocolloid 9 is covered by a flexible film layer (film) 25, preferably such that the film 25 extends beyond the hydrocolloid end. The film has a film upper surface area 27 and a film lower surface area 29 constituting a film thickness 31 and a film end 33. Further, the film preferably has an adhesive layer 35 adhered to the film lower surface area. The adhesive layer may be continuous or discontinuous along the film lower surface area. The adhesive layer has an adhesive upper surface area 37 and an adhesive lower surface area 39 constituting an adhesive thickness 41, and an adhesive end 43.

Design

The bandage preferably dimensioned in height, width and depth so as to provide enhanced adhesion and patient comfort.

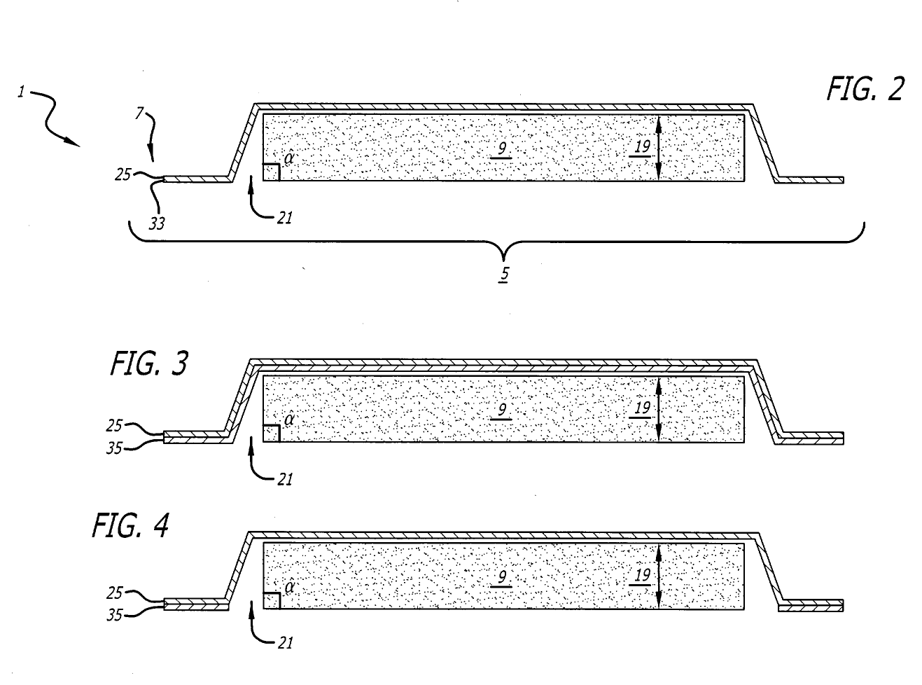

In one embodiment, the bandage 1 has the following configuration (FIG. 2). The bandage 1 preferably has a hydrocolloid region 9 wherein the hydrocolloid upper surface area 11 and hydrocolloid lower surface area 15 are substantially parallel to one another, and the hydrocolloid thickness 19 is substantially uniform. Further, the hydrocolloid end 21 is preferably substantially perpendicular (about 90°) to the hydrocolloid upper and lower surface areas 11/15. The bandage 1 also preferably has a film 25 formed such that the film 25 covers the hydrocolloid region 9 and extends beyond the hydrocolloid end 21 to form a film end 33. Preferably the film end extends beyond the hydrocolloid end. Thus in this embodiment, the bandage lower surface area 5 is formed from the hydrocolloid lower surface area 15 and the film lower surface area 29 extending beyond the hydrocolloid end 21.

The bandage may also preferably have an adhesive layer 35 situated between the film lower surface area 29 and the hydrocolloid upper surface area 11 (FIG. 3). The adhesive 35 is preferably formed such that the adhesive 35 covers the hydrocolloid region 9 and extends beyond the hydrocolloid end 21 to form an adhesive end 43. Thus in this embodiment, the bandage lower surface area is formed from the hydrocolloid lower surface area 15 and the adhesive lower surface area 39 extending beyond the hydrocolloid end 21. Alternatively, the adhesive 35 may be present discontinuously on the film lower surface area 29 (FIG. 4).

In one specific embodiment of the bandage, the dimensions may be as follows. The bandage may be of rectangular shape of about 1-2″ wide and about 1.5-3″ long measuring from bandage end to end. The film thickness may be about by about 1-2 mils, and the adhesive thickness about 1-3 mils. The hydrocolloid region may have a uniform thickness of about 20-65 mils, wherein the hydrocolloid end is a length of the same 20-65 mils. The film may have a length of about 1-2″ wide and about 1.5-3″ long, and extend beyond the hydrocolloid end by about 5 mm.

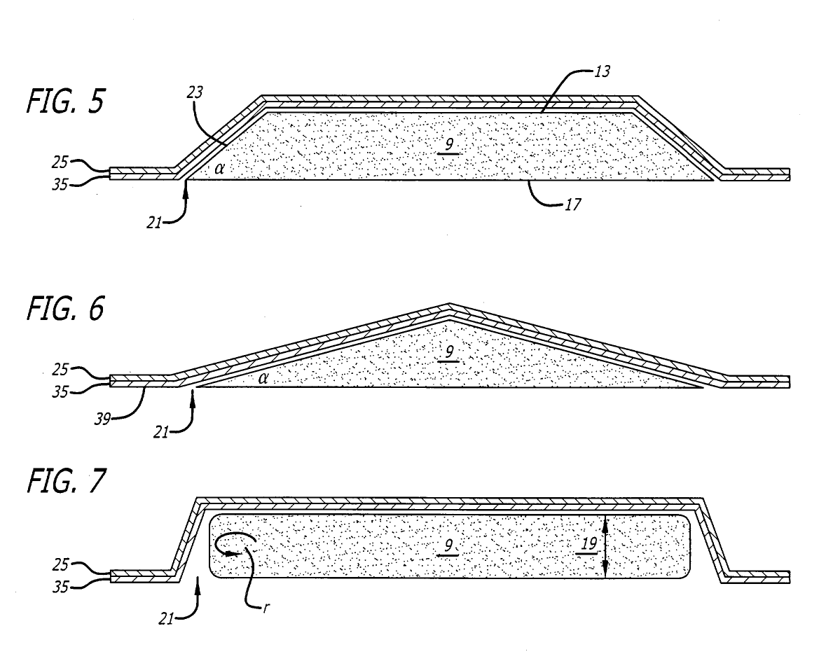

In an alternate embodiment, the bandage 1 has the following configuration (FIG. 5). The bandage 1 preferably has a hydrocolloid region 9 wherein the hydrocolloid upper surface area 11 and hydrocolloid lower surface area 15 are substantially parallel to one another. Further, the length of the hydrocolloid lower surface area 17 exceeds that of the hydrocolloid upper surface area 13, and the hydrocolloid end is formed at an angle α of preferably less than about 90° to the hydrocolloid lower surface area 15, and most preferably at an angle α of about 100 to about 60°. The bandage 1 also preferably has film 25 and adhesive layers 35 formed such that they cover the hydrocolloid region and extend beyond the hydrocolloid end 21.

In one specific embodiment of the bandage, the dimensions may be as follows. The bandage may be of rectangular shape of about 1-2″ wide and about 1.5-3″ long measuring from bandage end to end. The film thickness may be about by about 1-2 mils, and the adhesive thickness about 1-3 mils. The hydrocolloid region may have a non-uniform thickness, and at the greatest hydrocolloid thickness be about 20-65 mils, and wherein the hydrocolloid end has a length greater than the greatest hydrocolloid thickness. The ratio of the lower surface area length (17) over the upper surface area length (13) is in a range of 1.1-15. The film may have a length of about 1-2″ wide and about 1.5-3″ long, and extend beyond the hydrocolloid end by about 5 mm.

In another alternate embodiment, the bandage 1 has the following configuration (FIG. 6). The bandage 1 preferably has a hydrocolloid region 9 wherein the hydrocolloid upper surface area 11 and hydrocolloid lower surface area 15 are substantially not parallel to one another. Preferably, the hydrocolloid upper surface area length exceeds 13 that of the hydrocolloid lower surface area length 17, and the hydrocolloid upper surface area 15 extends directly to the lower surface area 11 at an angle α less than about 30°, preferably at an angle of about 1-10°, and most preferably at an angle of about 1-5°. The bandage also preferably has film 25 and adhesive 35 layers formed such that they cover the hydrocolloid region 9 and extend beyond the hydrocolloid end 21. Thus in this embodiment, the bandage lower surface area 5 is formed from the hydrocolloid lower surface area 15 and the adhesive lower surface area extending beyond the hydrocolloid end 21.

In one specific embodiment of the bandage, the dimensions may be as follows. The hydrocolloid region may have a non-uniform thickness, and at the greatest hydrocolloid thickness be about 20-65 mils, and wherein the hydrocolloid upper surface area length is greater than the hydrocolloid lower surface area length. The film may have a length of about 1-2″ wide and about 1.5-3″ long, and extend beyond the hydrocolloid end by about 5 mm.

In another alternate embodiment, the bandage 1 has the following configuration (FIG. 7). The bandage I preferably has a hydrocolloid region 9 wherein the hydrocolloid upper surface area 11 and hydrocolloid lower surface area 15 are substantially parallel to one another. Further, the hydrocolloid end 21 is preferably is not linear (angular or radius of curvature “r”) between the hydrocolloid upper 11 and lower 15 surface areas. The bandage 1 also preferably has a film 25 formed such that the film covers the hydrocolloid region 9 and extends beyond the hydrocolloid end. The bandage 1 also preferably has film 25 and adhesive layers 35 formed such that they cover the hydrocolloid region 9 and extend beyond the hydrocolloid end 21.

In one specific embodiment of the bandage, the dimensions may be as follows. The hydrocolloid region may have a non-uniform thickness, and at the greatest hydrocolloid thickness be about 20-65 mils, and wherein the hydrocolloid upper surface area length is about the same as the hydrocolloid lower surface area length. However, the hydrocolloid end is substantially non-linear having a radius of curvature being about 0.5-3″ or outer diameter (OD) of about 1-6″, most preferably is about 1″-3″. The film may have a length of about 1-2″ wide and about 1.5-3″ long, and extend beyond the hydrocolloid end by about 5 mm.

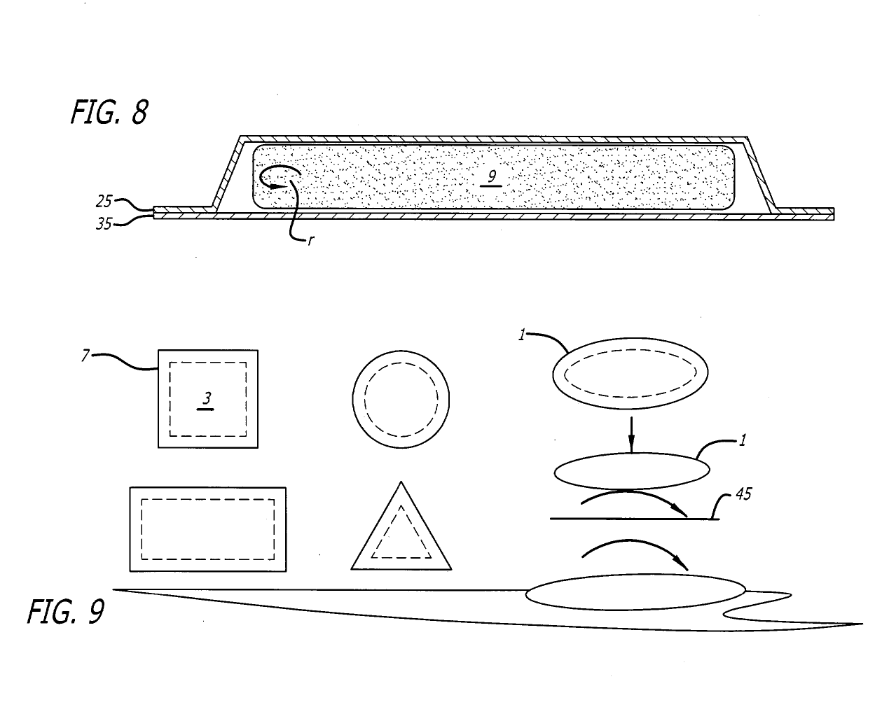

Alternatively or in addition, the bandage 1 may have an adhesive layer 35 affixed to the hydrocolloid lower surface area 15. Thus in this embodiment, the bandage lower surface area 15 is formed from the adhesive lower surface area 39 only (FIG. 8).

Flexible Film Layer The film materials which are useful for this invention are not particularly limited as long as they can provide a suitable substrate for the adhesive and/or hydrocolloid and are sufficiently strong to withstand removal from the skin and maintain its integrity, having been secured to the skin by the adhesive and hydrocolloid. Preferably, the backing film layer is water impervious.

The film is preferably flexible from the viewpoint of comfort. The flexibility is achievable by elasticity in any one or all axes of the material. Further, the film is preferably pliable to accommodate skin contours, when applied to areas of skin having alterations in surface angles. The film is also preferably breathable and conformable.

As is appreciated by those skilled in the art, the film materials may include, polyolefins (such as low, medium of high density polyethylene homopolymer, polypropylene or polyethylene or copolymers or blends of polypropylene or polyethylene, for example), ethylene vinyl acrylate, ethylene acrylic acrylate, ethylene methyl acrylate, polyvinyl chloride, polyester, polyurethane, polyamide and copolyester films, or combinations thereof. In one example, a film may include polyurethane and copolyester films having a moisture vapor transmission rate (MVTR) in a range of about 100 to about 5000 g/24 h m² (per ASTM E96 desiccant method with upright cup at 37° C.). A preferred film has a MVTR of greater than about 500 g/24 h m². Typical examples are Bioflex 125 (polyurethane) and Bioflex 235 (copolyester) available from Scapa Medical, Windsor Conn.

The film layer is also preferably of a thickness to provide sufficient strength to the bandage, but also of a thinness which will be comfortable to the wearer and pliable to contact all skin surfaces. In one embodiment, the film thickness is about 0.5 to about 10 thousands of an inch (mils), and in other embodiments, the film thickness is about 1 to about 5 mils, and most preferably about 1-2 mils. The film may or may not be of a uniform thickness over its length.

The film layer may be physically perforated to create micro or small openings and holes, which can improve the moisture permeation rate. Such film layer holes may extend from the film upper surface area through the film thickness to the film lower surface area.

Hydrocolloid Region The hydrocolloid region is comprised of any hydrocolloid composition known or developed by those skilled in the art, preferably having hydrophilic particles capable of swelling in water and transporting water. Hydrophilic particles which may be used in the invention include, but are not limited to naturally derived substances (such as silica, collagen, pectin, gelatin, starches, guar gum, gum arabic, locust bean gum, gum karaya, alginic acid and its sodium or calcium salts) and synthetic substances (such as such as sodium carboxymethylcellulose (CMC), crosslinked sodium carboxymethylcellulose, crystalline sodium carboxymethyl cellulose, polyvinyl alcohol, polyvinyl pyrollidone, high molecular weight polyethylene glycols and polypropylene glycols, cross-linked dextran and starch-acrylonitrile graft copolymer, starch sodium polyacrylate, gluten, polymer of methyl vinyl ether and maleic acid and derivatives; polyvinyl pyrrolidone, polyethylene glycols, polypropylene glycols, metal and/or ammonium salts of polyacrylic acid and/or its copolymers, and metal or ammonium salts of polystyrene sulfonic acid) or a variety of alternative commercially available absorbent products.

Adhesive Layer An adhesive useful in this invention is any substance which has good adhesion with the hydrocolloid region and/or the bandage in contact with the skin. The adhesive layer can be located on any part of, or the entirety of, the film lower surface area.

A wide range of adhesive materials can be used for the hydrocolloid dressing, and can be selected to maximize adhesion, absorption and comfort, while minimizing irritation to the user. The adhesive layer is preferably efficient at adhering to, but not damaging to the dermis or wound site. The adhesive layer further preferably has a relatively greater adherence to the film than to the dermis or wound site. There can be a desired range of adhesive strength for the adhesive layer in the present invention. The strength can vary relative to the selected use of the bandage.

The adhesive is preferably comprised of a polymeric adhesive composition. In one preferred embodiment, the polymeric adhesive composition comprises a pressure sensitive polymer mixture. In some embodiments, rubber based, polyacrylate based, urethane based or silicone based pressure sensitive adhesives can be used. Typical adhesives for use in the invention include, but are not limited to those physically or chemically crosslinked by polymer phase separation. Such phase separation is preferably not compromised at below 50° C. A typical pressure sensitive adhesive with good adhesion to skin is an acrylic PSA crosslinked thermally or cured through metal chelating agent such as aluminum acetoacetonate.

The adhesive layer thickness is preferably thick enough to afford suitable adhesion to and absorption from the dermis or wound site. In one embodiment, the adhesive layer thickness is about 0.5 to 8 mils, and in other embodiments, the thickness of the adhesive is about 0.5 to about 4 mils, and preferably about 1 to about 3 mils. The adhesive layer may or may not have a uniform thickness throughout its length.

Additives. Further, the hydrocolloid and/or adhesive layer can also contain additives, such as tackifiers, plasticizers and/or stabilizers to achieve the desired adhesive properties.

In some embodiments, the bandage (hydrocolloid and/or adhesive layer) can include therapeutic agents as additives, including those which can assist with wound protection and healing, such as alcohol, peroxide or betadine; antimicrobials; antibacterials, such as Triclosan, or polysporin; antivirals, such as Nonoxyl-9; antifungals, such as imidazole; antinflamatories such as hydrocortizone; wound healing promoters, such as growth factors; collagen; moisturizers, such as aloe or vitamins A, D or E; anti-scaring medications such as cortisone or pharmacologically active agents, including, but not limited to, analgesics, anesthetics, anti-inflammatories, and steroids. During processing of the bandage, agents may be combined with either the adhesive composition, with the hydrocolloid, or both, for example. In another example, the active agent may be adhered to at least a portion of the hydrocolloid or adhesive lower surface area.

Release Liner A release liner 45 can be applied to the bandage lower surface area 5 to facilitate to protect the adhesive 35 and/or hydrocolloid 9 before application of the bandage to the user, for example. Suitable liner materials include, but are not limited to bleached Kraft paper, silicone coated on one side at least where contact with the hydrocolloid and/or adhesive layer is made.

The liner can be of the same dimensions as the bandage, or can be of different dimensions to facilitate removal of the liner from the bandage. Where the liner is of different dimensions as the dressing, the liner can be larger in any one or all planar dimensions than the dressing. Further, the liner can have lines of weakness, such as scores or perforations, so as to facilitate removal of the liner from the dressing.

Bandage Shape

The bandage can generally be made in a variety of shapes. Examples of the shapes of the bandage include, but are not limited to a triangle, square, rectangle, circle or oval. Such shapes may be suitable for use on different regions of the body (FIG. 9). For example, the bandages are preferably a rectangular bandage about 1″-2″ wide and about 1.5″-3″ long along the bandage periphery. In another example, the bandage is preferably oval being about 1-2″ wide and 2-4″ long.

Color

In some embodiments, at least one of the hydrocolloid, film or/and adhesive are substantially transparent or clear, a flesh-like color or shade so as to effectively blend with the skin of wearer, or translucent. In other embodiments, the film is effectively colored or rendered ornate or patterned on its upper surface area.

Using the Bandage

To use the bandage a user obtains the bandage, removes the liner (if present) to expose the adhesive lower surface area and hydrocolloid lower surface area of the bandage, applying the bandage to the skin, having a wound with the lower surface of the bandage to the skin, such that the adhesive and hydrocolloid lower surface areas are preferably in direct contact with the skin (FIG. 9). The user then leaves the bandage on the wound site a proscribed period of time, such that the bandage absorbs moisture away from the wound, while maintaining adherence to the skin, even at the perimeter of the bandage (film end).

Alternatives

The bandage may be designed in alternate ways and remain within the spirit of this invention.

In one example, a bandage may be formed having a water absorbent hydrocolloid region with a thickness and an upper surface area and a lower surface area and a hydrocolloid end extending between the upper surface area to the lower surface area. Further, a flexible film layer may be affixed to the hydrocolloid upper surface area having a film thickness, and the film may be formed such that the film extends over the hydrocolloid upper surface area and at least beyond the hydrocolloid end. Optionally the bandage may have a removable liner on the hydrocolloid lower surface area. Preferably this bandage of would have a film thickness greater than about ¼ of the sum of the hydrocolloid thickness and the film thickness.

In a second example, a bandage may be formed having a hydrocolloid region for contacting a wound site, and a flexible film layer covering one side of the hydrocolloid region; and the bandage may include a bandage region extending outwardly beyond the hydrocolloid region at a selected distance of less than 5 mm, wherein the bandage region is substantially devoid of hydrocolloid. Preferably a bandage of this type would also include a liner removably attached to a side of the hydrocolloid region opposite from the flexible film layer. The bandage region may extend from the hydrocolloid end for any distance including less than 5 mm or more than 5 mm.

In a third example, the bandage may have wells or dimples formed in any one of or all of the film, adhesive or hydrocolloid thickness, such that the thickness has areas of greater and lesser thickness. Further, the bandage may have internal voids, such that any one of the layer surface areas is not in direct apposition to the surface of another. For example, a void may be created between the film layer and the hydrocolloid such that the film lower surface area is not in direct contact with the hydrocolloid upper surface area at some points over the length of the bandage. Alternatively, an internal void may be created in the hydrocolloid thickness, such that between the hydrocolloid upper surface area and lower surface area, there is region of the hydrocolloid thickness devoid of hydrocolloid.

What is claimed is:

1. A hydrocolloid bandage for use on a wound site comprising at least:
a hydrocolloid region, wherein the hydrocolloid region has a hydrocolloid upper surface area having an upper surface area length and a hydrocolloid lower surface area having a lower surface area length, and wherein the hydrocolloid region has a hydrocolloid end extending for an end length between the hydrocolloid upper surface area and the hydrocolloid lower surface area; and
a film layer adhered to the hydrocolloid upper surface area and hydrocolloid end length, the film layer having a film length and a film lower surface area, wherein the film layer has a film length which exceeds the upper surface area length and hydrocolloid end length, and wherein the film layer extends beyond the hydrocolloid end such that a bandage lower surface area is formed by the hydrocolloid lower surface area and the film lower surface area extending beyond the hydrocolloid end to a film end.

2. A hydrocolloid bandage for use on a wound site comprising at least:
a hydrocolloid region, wherein the hydrocolloid region has a hydrocolloid upper surface area having an upper surface area length and a hydrocolloid lower surface area having a lower surface area length, and wherein the hydrocolloid region has a hydrocolloid end extending for an end length between the hydrocolloid upper surface area and the hydrocolloid lower surface area; and
an adhesive layer film layer adhered to the hydrocolloid upper surface area and hydrocolloid end length having an adhesive layer length and an adhesive layer upper surface area, wherein the adhesive layer length exceeds the upper surface area length and hydrocolloid end length, and wherein the adhesive layer extends beyond the hydrocolloid end such that a bandage lower surface area is formed by the hydrocolloid lower surface area and the adhesive lower surface area extending beyond the hydrocolloid end to an adhesive end; and
a film layer adhered to the adhesive layer upper surface area.

3. The hydrocolloid bandage of claim 1, wherein the bandage further comprises an adhesive layer adhered to the bandage lower surface area.

4. The hydrocolloid bandage of claim 1, 2 or 3, wherein the hydrocolloid upper surface area and the hydrocolloid lower surface area are substantially parallel, and wherein the hydrocolloid end is at an angle substantially perpendicular to the hydrocolloid upper and lower surface areas.

5. The hydrocolloid bandage of claim 4, wherein the hydrocolloid upper surface area and the hydrocolloid lower surface area are substantially parallel, and wherein the hydrocolloid end is linear.

6. The hydrocolloid bandage of claim 4, wherein the hydrocolloid upper surface area and the hydrocolloid lower surface area are substantially parallel, and wherein the hydrocolloid end has a radius of curvature of 1″-3″.

7. The hydrocolloid bandage of claim 1, 2 or 3, wherein the hydrocolloid upper surface area and the hydrocolloid lower surface area are substantially parallel, and wherein the hydrocolloid lower-surface area length exceeds the hydrocolloid upper surface area length, and wherein the hydrocolloid end is at an angle of less than about 90° to the hydrocolloid lower surface area.

8. A hydrocolloid bandage for use on a wound site comprising at least:
a hydrocolloid region, wherein the hydrocolloid region has a hydrocolloid upper surface area having an upper surface area length and a hydrocolloid lower surface area having a lower surface area length, and wherein the hydrocolloid region has a hydrocolloid end between the hydrocolloid upper surface area and the hydrocolloid lower surface area;
the hydrocolloid region having the hydrocolloid upper surface area and hydrocolloid lower surface area are substantially not parallel to one another, and further wherein the hydrocolloid upper surface area length exceeds that of the hydrocolloid lower surface area length, and wherein the hydrocolloid upper surface area extends directly to the lower surface area at an angle of less than about 60°; and
a film layer adhered to the hydrocolloid upper surface area, the film layer having a film length and a film lower surface area, wherein the film layer has a film length which exceeds that of the upper surface area length, and wherein the film layer extends beyond the upper surface area length.

9. The hydrocolloid bandage of claim 1 or 2 wherein the film layer extends from the hydrocolloid end for a distance of about 5 mm.

10. A hydrocolloid bandage of claim 1, wherein the adhesive layer is about 0.5 to about 8 mils.

11. A hydrocolloid bandage of claim 1, wherein the film layer is about 0.5 to about 10 mils.

12. A hydrocolloid bandage of claim 1, further comprising a release liner adhered to the bandage lower surface area.

13. A hydrocolloid bandage of claim 1, wherein at least one of the adhesive layer, film layer, or hydrocolloid region are substantially transparent or clear.

14. A hydrocolloid bandage of claim 1, wherein at least one of the adhesive layer, film layer, or hydrocolloid region are substantially flesh colored.

15. A method of using the hydrocolloid bandage of claim 1 comprising obtaining the bandage and applying the bandage lower surface area to a user's skin or wound site.

16. A bandage comprising a water absorbent hydrocolloid region having a thickness and an upper surface area and a lower surface area and a hydrocolloid end extending between the upper surface area to the lower surface area,
a flexible film layer affixed to the hydrocolloid upper surface area having affixed, wherein the flexible film layer has a film thickness and the film is formed such that the film extends over the hydrocolloid upper surface area and at least beyond the hydrocolloid end; and
optionally a removable liner on the hydrocolloid lower surface area.

17. A bandage of claim 16, wherein the film thickness is greater than ¼ of the sum of the hydrocolloid thickness and the film thickness.

18. A bandage comprising a hydrocolloid region for contacting a wound site, and a flexible film layer covering one side of the hydrocolloid region; and including a bandage region extending outwardly beyond the hydrocolloid region at a selected distance, of less than about 5 mm, the bandage region being devoid of hydrocolloid.

19. A bandage of claim 18, further comprising a liner removably attached to a side of the hydrocolloid region opposite from the flexible film layer.

20. A bandage of claim 18, wherein the selected distance is less than about 5 mm.

21. A bandage of claim 18, wherein the selected distance is greater than about 5 mm.
